(19) United States
(12) Patent Application Publication
(10) Pub. No.: US 2018/0067228 A1
(43) Pub. Date: Mar. 8, 2018

(54) METHOD AND DEVICE FOR UNDERWATER DETECTION OF PHYSICOCHEMICAL PARAMETERS FOR IDENTIFYING HYDROCARBON RESERVOIRS

(71) Applicant: Eni S.p.A., Roma (IT)

(72) Inventors: Micaela NALI, Milano (IT); Stefano CARMINATI, Monza (IT); Francesco GASPARONI, Venezia (IT)

(21) Appl. No.: 15/563,184

(22) PCT Filed: Mar. 31, 2016
(86) PCT No.: PCT/IB2016/051841

§ 371 Date: Sep. 29, 2017

(30) Foreign Application Priority Data
Mar. 31, 2015 (IT) MI2015A000462

(51) Int. Cl. G01V 11/00; G01N 33/18; G01N 33/24; G01V 1/38; B63G 8/00

(52) U.S. Cl. CPC G01V 11/002; G01N 33/1886; G01N 33/241; B63G 2008/004; G01V 1/3835; B63G 8/001; G01V 1/3808

(57) ABSTRACT

The present invention relates to a method and to a device for in situ underwater detection of physicochemical parameters in a body of water with a particular focus on using the acquired data for prospecting for potential reservoirs rich in hydrocarbons. The present invention provides a method for in situ real-time underwater detection of physicochemical and optionally biological parameters in a body of water by using an instrumented detection carrier. The present invention secondly provides an underwater instrumented detection carrier for in situ real-time detection of physicochemical parameters in a body of water comprising an autonomous underwater vehicle (AUV) arranged to use an instrumented module (or “payload”), capable of completing programmed missions without human intervention and configured to carry out the method provided by the invention.

The present invention relates to a method and to a device for in situ underwater detection of physicochemical parameters in a body of water with a particular focus on using the acquired data for identifying potential hydrocarbon reservoirs.

The technique of analysing surface occurrences of hydrocarbons dates back to the pioneering stages of petroleum exploration and the first wells were drilled close to occurrences which were visible to the naked eye (macro-occurrences or macro-seepage). Developments in the technologies used in exploration, in particular in geophysics, and the exhaustion of points adjacent to such macro-occurrences has led to this approach having been neglected for many years. An appreciable improvement in analytical instrumentation has given a new lease of life to surface geochemical prospecting techniques, because invisible occurrences (micro-occurrences or micro-seepage) can now be detected, even with great accuracy. Numerous initiatives and research projects have thus been started with the aim of developing methods which would enable reliable acquisition and interpretation of the data associated with surface micro-occurrences. Surface geochemical surveys are used to identify the existence of possible underground hydrocarbon reservoirs on the basis of determining the presence, composition and quantity of the emissions which reach the surface. This kind of survey can be carried out using instruments and methods which are applied as a function of the conditions under which work is carried out. The greatest differentiating factor is the environment in which work is carried out (terrestrial or underwater). In the case of surveys carried out in an underwater environment, the current method involves sampling portions of sediments originating from the seabed (seabed core sampling) using various devices depending on the nature of the sea floor (“piston corer”, “vibro corer” or “gravity corer”). The designated sampling points must take account of the presence of faults/fractures in the seabed which might facilitate migration of hydrocarbons from the reservoir to the surface to form non-negligible occurrences (macro-occurrences). The samples are then collected and taken on board an appropriately equipped support vessel and subsequently (generally on completion of the sampling campaign) transferred to land and sent to suitably equipped laboratories where the analysis step is carried out.

The drawbacks related to prior art methods may primarily be found in the sampling procedures. The core sampling programme normally focuses on carrying out sampling which is intended to sense geochemical and microbiological parameters in areas where there is the greatest probability of finding hydrocarbon occurrences, which appreciably reduces the number of samples acquired and complicates the interpretation stage which must take account of the correlation between the position of the surface macro- or micro-occurrences and the actual position of the origin of the occurrences, i.e. the reservoir which released them. The laboratory analysis step is time-consuming and means that the prospecting results are only available after a relatively long period of time (sometimes months), thus appreciably delaying the actions arising from the consolidation of the results obtained from the core sampling campaign. Furthermore, in the event of results which are not unambiguously interpretable it is often difficult and costly to return to the survey point to repeat the sampling and subsequent analyses. A further drawback related to the methods described in the prior art is that, in some situations, such as shallow waters and unfavourable logistic conditions, core sampling operations as they are normally designed may be difficult, if not impossible, to carry out.

The object of the present invention is to provide a method and a device for in situ underwater detection of physicochemical parameters which overcome the drawbacks of the prior art.

The present invention provides an underwater instrumented carrier for in situ real-time detection of physicochemical parameters in a body of water, comprising an autonomous underwater vehicle (AUV), an instrumented module (or “payload”), able to complete programmed missions without human intervention and configured to carry out the method described below.

A second object of the present invention is a method for in situ real-time underwater detection of physicochemical parameters in a body of water by using an instrumented carrier comprising the steps described in detail below.

Further features of the invention are defined by the dependent claims which are an integral part of the present description.

The features and advantages of the present invention will emerge clearly from the following description of a non-limiting exemplary embodiment thereof with reference to the figures of the attached drawings, in which:

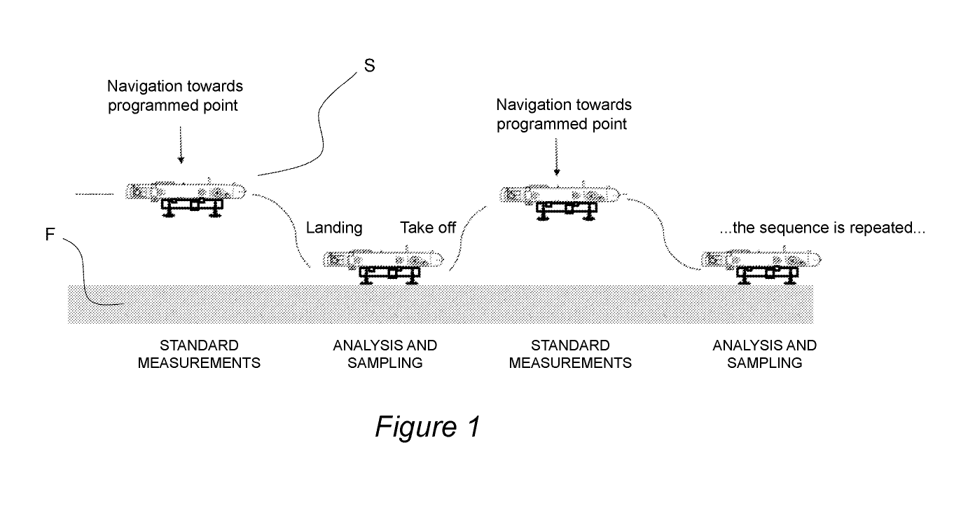

FIG. 1 is a schematic representation of an instrumented carrier (S) which moves in a body of water along a programmed detection path T and carries out the steps of the method provided by the invention;

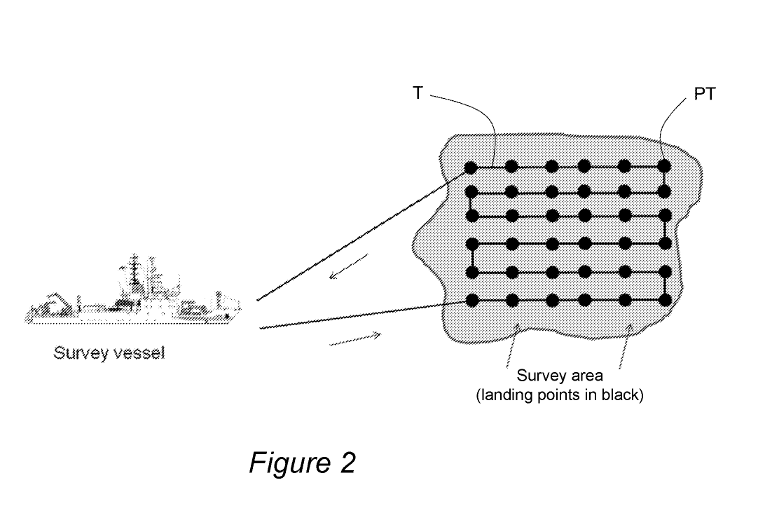

FIG. 2 shows an example of a detection path T with defined measuring points PT close to the bed F of the body of water with optional landing on said bed;

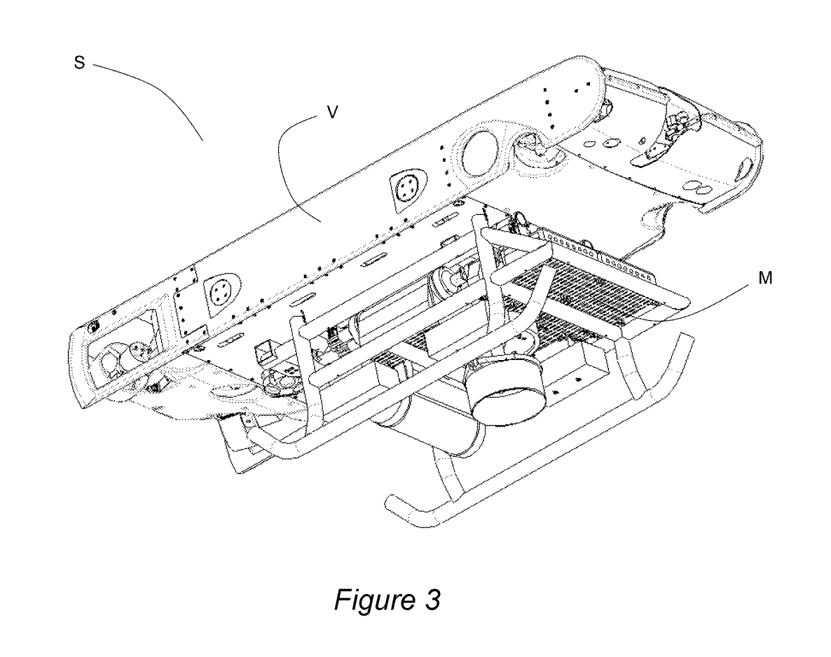

FIG. 3 shows an axonometric view of an exemplary embodiment of the instrumented carrier (S) for the detection, with some components removed for clarity, with identification of the carrier (V) and the instrumented module M (payload);

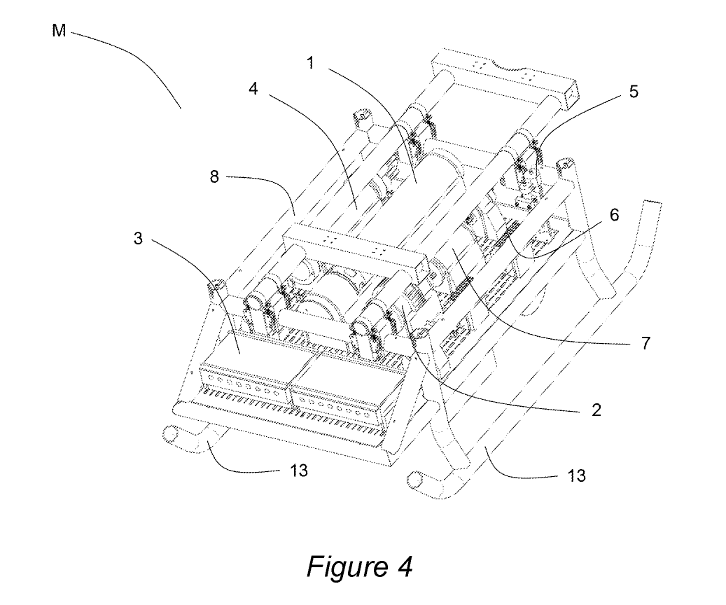

FIG. 4 shows an axonometric view of an exemplary embodiment of the instrumented module M (payload), with some components removed for clarity;

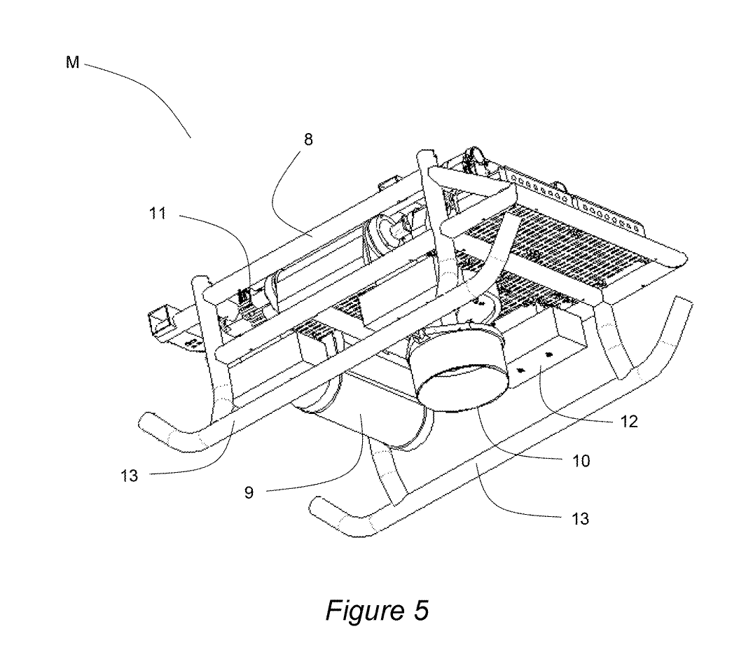

FIG. 5 shows a different axonometric view of an exemplary embodiment of the instrumented module M (payload), with some components removed for clarity.

With reference to FIGS. 3, 4, and 5, the present invention provides an underwater instrumented carrier module (S) for the detection which is capable of implementing the steps of the method described below.

With reference to FIG. 3, in a preferred configuration, the instrumented carrier module comprises two main components: an underwater carrier (V), preferably an autonomous underwater vehicle (AUV), and an instrumented module M. The carrier (V) has the function of transporting the instrumented module M along a path T and to predetermined points PT of the mission and of permitting the operation thereof in accordance with the specific requirements, for example by setting it down on a bed F of a body of water or at a predetermined distance from said bed, maintaining it at said point for the time required to carry out the planned operations, for example carrying out sampling and measurement cycles, and transferring it to the subsequent point once the operations at the previous point are complete. The carrier (V) does not require any kind of physical link (e.g. umbilical cable) with the operator or with the support vessel in order to operate. The carrier (V) has the ability to move autonomously once it has been launched (from a ship, from land or from another platform) in order to reach the various programmed points PT of the mission, position the instrumented module M at each point, maintain it substantially stationary for the time required and finally return to the point specified for recovery. To this end, the carrier (V) is equipped with an on-board propulsion, navigation and power supply system. The carrier (V) is configured to accommodate the instrumented module M (payload) comprising an assembly of devices such as sensors, analysers and samplers, favourably selected in relation to the activity to be carried out. In a preferred configuration, the instrumented module M accommodates the subsystems required to operate the installed instrumentation, such as the acquisition and control electronics, data storage, actuation systems, sensors for monitoring technical parameters of the system (for example attitude, integrity, status) and the interfaces with the carrier module (V). In a preferred embodiment, the carrier (V) is an autonomous underwater vehicle (AUV) of the hybrid type equipped with from 4 to 8 thrusters which permit movement, hovering, station-keeping and the ability to land on and take off from the bed F of a body of water. In a further preferred embodiment, the instrumented module M (FIG. 4) comprises a plurality of sensors for monitoring parameters of interest and pressure vessels containing the control electronics 7 of the sampling system, the control electronics 4 of the instrumented module and the valve block 6 which directs the sampling and the subsequent treatment of the sample, which has been taken, for example towards the in situ analytical instrumentation or towards storage for subsequent laboratory analysis. Said module M is furthermore equipped with a main supporting structure 8 capable of setting the instrumented carrier (S) down on the bed F of the body of water. The structure 8 comprises appropriate supporting means for this purpose. For the purposes of the present invention, supporting means are taken to be structures which allow the instrumented carrier (S) to land on the bed F and which withstand the loads arising from the operating conditions of said structures. In a preferred embodiment of the invention, said seabed supporting means are embodied by the skids 13 (FIGS. 4 and 5).

In a further preferred embodiment of the instrumented module M, with reference to FIGS. 4 and 5, the installed instrumentation comprises:
an underwater quadrupole mass spectrometer 1 for analysing light hydrocarbons and other chemical species, equipped with a device 2 (cryotrap) for separating the water vapour from the sample;
an underwater gamma spectrometer 9 for analysing radionuclides;
a sensor for dissolved methane 5 equipped with a dedicated pump;
a benthic chamber 10 capable of confining a volume of water and permitting the measurement of low concentration levels at the water-sediment interface;
a syringe sampler 3 for taking discrete quantities of liquid to carry out comparative laboratory analyses;
a pumping system 11 equipped with peristaltic pumps.

The instrumented module M is also equipped with foam floatation blocks 12.

A second object of the present invention is a method for in situ underwater detection of physicochemical parameters which provides an effective alternative to conventional techniques for the detection seabed hydrocarbon occurrences which use “sea bottom coring” sampling techniques with subsequent laboratory analyses. There are numerous advantages pursuing the approach of measuring underwater physicochemical parameters, preferably close to the bed F of the body of water, with the aim of identifying potential hydrocarbon reserves, such as a greater availability of data which are not tied to the number of core samples taken, the possibility to have the results available in a very short time, the possibility to repeat measurements in areas or at points where inconsistency in results might make it necessary. Furthermore, getting rid of the equipment and means required for underwater core sampling is an undoubted advantage in logistical terms when working in areas where conventional techniques would be difficult to use (for example shallow waters with inadequate draught for conventional means, places difficult to access with the means used in the prior art).

With reference to FIGS. 1 and 2, the method provided by the present invention makes it possible to sense physicochemical parameters of interest in a body of water not only during the step of navigating along the programmed path T, but implements a further characteristic function which is that of being able to stop an instrumented carrier (S) at precise and predetermined positions PT relative to the bed F of the body of water, maintaining the means substantially stationary and carrying out spot measurements at programmed distances from the bed F of the body of water. Stationary positioning of the instrumented carrier (S) should be taken to mean substantially maintaining a programmed position in the body of water taking into account disruptive effects, such as water currents. This detection method permits accurate geolocation of the measured parameters, thus they may be better matched up with the emission sources of said parameters. To this end, the method is implemented by using an instrumented carrier (S) (FIG. 3). Measurement of physicochemical parameters in the body of water is more accurate and advantageous if carried out close to the bed F, preferably if the water sample originates from the interface between the bed F and the body of water and more preferably if said sample originates from interstitial water taken from the bed F.

The method for in situ real-time underwater detection of physicochemical parameters in a body of water by means of an instrumented carrier (S) comprises the steps of:
programming the instrumented carrier (S) to follow a path (T) along which detection operations have to be performed, to this end the instrumented carrier (S) is configured to be programmed to carry out missions which include moving along a defined path;
programming a plurality of measuring points PT along said path T, to this end the instrumented carrier (S) is capable of reaching the programmed detection points PT by means of an on-board propulsion and navigation system;
defining a plurality of physicochemical parameters to be detected along the path T and at the programmed points PT; depending on the defined parameters, the instrumented carrier will be advantageously and selectively equipped with the instrumentation required to carry out the detection operations for said parameters;
launching the instrumented carrier (S) into the body of water;
causing the instrumented carrier (S) to navigate to the starting point of the programmed path T;
carrying out the detection of the defined parameters along the programmed path T by means of said instrumented carrier (S);
stopping the instrumented carrier (S) at the defined measuring points PT and maintaining said means substantially stationary for the time period required to carry out the programmed detection operations at a maximum distance of 2 m from the bed F of the body of water, more preferably at a maximum distance of 0.2 m; to this end the carrier (V) is equipped with instrumentation such as echo sounder, Doppler Velocity Log, Inertial Navigation System, pressure sensor, for accurate, real-time determination of distance and velocity relative to the bed F of the body of water.
carrying out the detection of the defined parameters at the programmed measuring points PT by means of said instrumented carrier (S);
resuming navigation with the instrumented carrier (S) along the programmed path T;
acquiring the data recorded by the instrumentation of the instrumented carrier (S);
processing and analysing the detected parameters.

With reference to FIGS. 1 and 3, in a preferred implementation the detection method comprises the step of stopping the carrier (V) at the defined measuring points PT and setting down the instrumented module M, mounted solidly on the carrier (V), on the bed F of the body of water during the measurement step of the defined parameters in such a way as to analyse a volume of water at the interface with the bed F and to ensure geo-referenced measurements, with minimised disturbance in the vicinity of the source, raising the instrumented module M back up on completion of the spot detection and resuming navigation along the programmed path T.

A further preferred implementation of the detection method comprises the step of isolating a volume of water close to the bed F of the body of water, preferably at the water-bed F interface of the body of water, on which the detection of the defined parameters has to be performed.

Detection of the defined parameters with the instrumented carrier (S) hovering and the instrumented module M leant on the bed F of the body of water and with the isolated volume of water at the water-bed F interface allows the measured data to be precisely located relative to the source, so minimising disruptive effects due to any water currents or other disturbances acting on said volume of water.

A preferred implementation of the detection method comprises using a benthic chamber to isolate the volume of water to be analysed.

The parameters to be detected are numerous and depend on the type of instrumentation installed on board the instrumented module M. In a preferred embodiment of the method, provided by the present invention, the parameters to be detected which are measured in the water are selected from the following:
hydrocarbons (presence, composition, quantity)
radionuclides (⁴⁰K, ²³⁸U, ²³²Th, ²²²Rd)
temperature
salinity
pH
redox potential
dissolved oxygen
dissolved methane
dissolved CO₂
dissolved nitrogen dissolved H₂S
dissolved He.

A further preferred implementation of the detection method includes the step of collecting interstitial water present in the bed F of the body of water in the case of soft beds, for example sandy beds; to this end, the method provides a system for sampling water present in the soft bed F of the body of water, preferably a syringe sampler. Such a sampler may optionally be used both for performing in situ analysis by means of the instrumentation installed on board the instrumented module M and for collecting water samples to be further analysed in specific laboratories.

The above-described detection method may include, in a preferred embodiment thereof, the step of collecting sediment samples from the bed F of the body of water to perform analyses which are not possible in situ, such as microbiological analyses with the aim of qualitatively and quantitatively determining the bacterial species which live by using hydrocarbons as their sole source of carbon. The results of the microbiological analyses consolidate and confirm the information obtained by the other survey methods.

A preferred embodiment of the method, provided by the present invention, is characterised in that the acquired data are processed and analysed, using per se known methods, in order to obtain prospecting data suitable for identifying geological formations rich in hydrocarbons. the use of the described method in presence of both macro-occurrences and micro-occurrences of hydrocarbons is of particular interest. The wide variety of data made available by the described method, the accuracy of the measurements and the precise location of sampling make it possible to carry out detailed analyses and enable the identification of hydrocarbon reservoirs.

The above-described method is characterised by programming a detection path T and measuring pointpoints PT located along said path at the start of the mission; in a preferred embodiment of method, the path T and/or number and/or position of the detection points PT may be autonomously and advantageously modified by the instrumented carrier (S) on the basis of the measured parameters selected for this purpose, thus establishing intelligent, adaptive behaviour of the detection method. Consequently, the detection grid set prior to launching the instrumented carrier (S) (FIG. 2) may be modified not only in terms of trajectory but also in terms of the density of transits per area and the number of the sampling pointpoints PT, on the basis of the feedback from the already measured data. Depending on the settings made in the programming step and on the basis of the measured parameters which are selected for the purpose of implementing the above-described intelligent, adaptive behaviour, the difference in absolute value of the measurement of said parameters relative to recorded reference threshold values (background values) in some detection areas may cause the instrumented carrier module (S) to modify its sampling plan by stepping up measurements in the vicinity of areas which are potentially of interest. The instrumented carrier (S) may thus modify the measurement path T, the number of and the measuring pointpoints PT, where the values of the parameters identified by the intelligent, adaptive function are appreciably different from the background value, since it may be assumed in such areas that there is a potential hydrocarbon reservoir. In order to implement such functionality, a preferred embodiment of the detection method provided by the present invention is characterised in that the previously described steps are carried out using as a carrier an autonomous underwater vehicle (AUV), arranged to use an instrumented module M (payload) able to complete programmed missions without human intervention, said vehicle being equipped with an on-board propulsion, navigation and power supply system.

It may thus be seen that the method and the device for underwater detection of physicochemical and optionally microbiological parameters for identifying hydrocarbon reservoirs according to the present invention achieve the objects stated above.

The method for underwater detection of physicochemical and optionally microbiological parameters and the associated instrumented carrier (S) of the present invention conceived in this manner may in any event be modified and varied in numerous ways, all of which fall within the same inventive concept; moreover, any details may be replaced by technically equivalent elements. In practice, the materials used, together with the shapes and dimensions, may be any as defined according to the technical requirements. The examples and lists of possible variants of the present application should be taken as non-exhaustive lists.

The scope of protection of the invention is thus defined by the attached claims.

1. A method for in situ real-time underwater detection of physicochemical parameters in a body of water by means of an instrumented detection carrier (S) comprising the steps of:
a) programming the instrumented detection carrier (S) to follow a path (T) along which the detection operations have to be performed;
b) programming a plurality of measuring pointpoints (PT) along said path (T);
c) defining a plurality of physicochemical parameters to be detected along the path (T) and at the programmed pointpoints (PT);
d) causing the instrumented detection carrier (S) to navigate to the starting point of the programmed path (T);
e) carrying out the detection of the defined parameters along the programmed path (T) by means of said instrumented detection carrier (S);
f) stopping the instrumented detection carrier (V) at the programmed measuring pointpoints (PT) and maintaining said means substantially stationary for the time period required to carry out the detection operations at a maximum distance of 0.2 m from the bed (F) of the body of water;
g) carrying out the detection of the defined parameters at the programmed measuring pointpoints (PT) by means of said instrumented detection carrier (S);
h) resuming navigation with the instrumented detection carrier (S) along the programmed path (T);
i) acquiring the data recorded by the instrumentation of the instrumented detection carrier (S).

2. A method according to claim 1, characterised in that the instrumented detection carrier (V) is stopped at the programmed measuring points (PT) and the instrumented module (M) is leant on the bed (F) of the body of water during the measurement step of the defined physicochemical parameters.

3. A method according to claim 1, in which the detected physicochemical parameters of the body of water are selected from the following:
hydrocarbons (presence, composition, quantity)
radionuclides (⁴⁰K, ²³⁸U, ²³²Th, ²²²Rd)
temperature
salinity
pH
redox potential
dissolved oxygen
dissolved methane
dissolved CO₂
dissolved nitrogen
dissolved H₂S
dissolved He.

4. A method according to claim 1, comprising the step of isolating a volume of water at the water-bed (F) interface of the body of water, on which the detection of the defined parameters has to be performed.

5. A method according to claim 1, comprising the step of using a benthic chamber (10) to carry out the detection of the defined parameters.

6. A method according to claim 1, further comprising the step of collecting interstitial water present in the bed (F) of the body of water.

7. A method according to claim 1, further comprising the step of collecting sediment from the bed (F) of the body of water.

8. A method according to claim 1, characterised in that the acquired data are processed and analysed in order to obtain prospecting data suitable for identifying geological formations rich in hydrocarbons.

9. A method according to claim 1, characterised in that the detection carrier (V) is an autonomous underwater vehicle (AUV) arranged to use an instrumented module (M), said vehicle being equipped with an on-board propulsion, navigation and power supply system.

10. A method according to claim 1, characterised in that the path (T) and/or the number and/or the position of the measuring points (PT) are autonomously modified with respect to what was originally programmed on the basis of the measured parameters which have been detected.

11. An underwater instrumented detection carrier (S) for in situ real-time detection of physicochemical parameters of a body of water comprising an autonomous underwater vehicle (V) and an instrumented module (M) configured to carry out the method described in claim 1.

12. An instrumented detection carrier (S) according to claim 11, in which the autonomous underwater vehicle (V) is of the hybrid type equipped with from 4 to 8 thrusters which permit movement, hovering, station-keeping and the ability to land on and take off from the bed (F) of the body of water.

13. An instrumented detection carrier (S) according to claim 11, in which the instrumented module (M) comprises support means.
